(19) United States
(12) Patent Application Publication
(10) Pub. No.: US 2002/0055171 A1
(43) Pub. Date: May 9, 2002

(54) PREPARATION OF BIOLOGICALLY ACTIVE 3-METHYLENEOXINDOLE AND DEFINITION OF ITS APPLICATION IN STIMULATION OF PLANT GROWTH AND TISSUE REPAIR

(76) Inventor: VIRENDA KUMAR TULI, PALOS VERDES, CA (US)

Correspondence Address: OWEN J. BATES, 777 WEST MIDDLEFIELD ROAD, #177, MOUNTAIN VIEW, CA, 94043, US

(21) Appl. No.: 09/087,235

(22) Filed: May 27, 1998

(51) Int. Cl. C12N 5/00; C12N 5/02

(52) U.S. Cl. 435/420

(57) ABSTRACT

Identification of the true nature and metabolic action of 3-methyleneoxindole (MO), a naturally occurring catabolite of the plant auxin, indole-3-acetic acid (IAA). Description of the two novel methods of synthesis of MO which produce the pure, biologically active auxin compound of MO. Discovery and description of the causes for the erroneous classification of MO as an inert compound with no auxin activity. These findings and methods of synthesis correct the ubiquitous theoretical errors which have driven scientific investigation and plant science research of plant auxins for nearly forty years.

Researchers have failed to observe the auxin activity of MO because of the use of standard but incorrect synthetic techniques which have consistently produced impure forms of 3-bromooxindole-3-acetic acid (3-Brox), the synthetic precursor of MO. In addition, the use of dimethylsulfooxide as a solvent for MO has been identified as a major source of contamination of MO during synthesis.

This invention describes two novel methods for synthesizing (MO). By using purified MO and avoiding the use of dimethylsulfoxide, it was found that the resulting MO product to be 100 to 1,00 fold more effective than IAA in promoting rooting in plant cuttings; supporting tissue differentiation and growth in stage I, stage II and stage III media used for the micropropagation of explants; promoting the formation of callus tissue over wounded plant parts; and, stimulating the production of interstitial tissue between scion and rootstock to increase the success rate for grafting. Therefore, this invention identifies MO as the dominant auxin in shoot acceleration, root development, wound sealing and scion acceptance.

Finally, the correct pathway from IAA to MO is presented.

### BACKGROUND

1. Field of Invention

This invention relates to the field of plant physiology; more specifically, the synthesis and isolation of plant metabolic regulators and the application of these substances in agriculture.

2. Discussion of Prior Art

The correlation of growth between one plant part and that same plant part on another plant was apparent to nineteenth century plant physiologists. Continuing investigations led to the discovery of the substances responsible for metabolic regulation in all plants. Direct lines of experimentation trace to the identification of a particular class of compounds that occur in very small quantities in plants and function as metabolic regulators. Compounds such as auxins, cytokinnins, gibberellins, abcisic acid and ethylene are currently considered the five hormones controlling plant growth. These hormones exert powerful effects when properly isolated and applied to plants. It is most probable that hormones bind with protein receptor sites in the cell to carry out their actions. Receptor sites are nearly as discriminating as are sites on enzymes that bind substrate molecules. One type seems to be on the plasmalemma and is involved in the transport of auxin in and out of the cell. Another auxin-binding protein is on the endoplasmic reticulum and appears to be involved in the reaction whereby auxin promotes growth. Critical to the successful utilization of plant hormones is the fidelity of the isolated hormone molecule to that structure defined by the receptor sites within the cells.

For example, high fidelity auxin molecules are more likely to produce the desired stimulatory effects on plant growth than molecules that are unstable or are near copies of the natural auxin.

Agricultural scientists and researchers have long considered and identified Indole-3-acetic acid (IAA) as the primary plant growth stimulant or auxin. However, using IAA in stimulating growth has proved difficult. IAA is an unstable compound and degrades readily in normal use, solutions of IAA must be used quickly and exhibit a short period of activity. As a more effective alternative to the natural substance, synthetic auxins have been developed. 3-Indolepropionic acid, 3-Indolebutyric acid, napthaleneacetic acid, 2,4-dichlorophenoxyacetic acid are synthetic auxins. They are commonly used instead of IAA because the latter is so unstable.

Unfortunately, some of the synthetic auxins are toxic (2,4-dichorophenoxyacetic acid) and their use is severely restricted by the Environmental Protection Agency (EPA). These substances are known to contaminate ground water, and some are identified as carcinogenic agents. Furthermore the substances do not degrade in the cell but accumulate in the plants and give rise to the contamination effects identified by the EPA. Consequently, legal restrictions make widespread use of the synthetic auxins difficult and identified effects make use dangerous.

Ideally, a naturally occurring, easily isolated or synthesized, and chemically stable auxin would be of great utility in the cultivation of plants as well as for use in developmental research in cell physiology. The research described in this document has been undertaken to demonstrate that (a) 3-methyleneoxindole (MO), a naturally occurring metabolite of IAA in higher plants, possesses auxin activity and (b) to develop a novel procedure to synthesize MO.

The instability of the IAA oxidation products has provided researchers with a formidable task in synthesizing pure products and in subsequently demonstrating their auxin activity. As a result, the current consensus holds that only IAA functions as the primary growth stimulant in plants.

Furthermore, it is believed firmly that the oxidation products of IAA possess no auxin activity. The research represented by this application has focused on the preparation of those previously known oxidation products of IAA, that have been believed to be devoid of any auxin activity.

The research described by this specification has successfully isolated the pure, active Photooxidation products of IAA. By means of this newly developed synthetic scheme which has been painstakingly elucidated over many years, pure and active forms of oxindole-3-carbinol (HMO) have been produced. It has been generally agreed that when IAA is oxidized it looses all biological activity and the compound is rendered useless for cellular regulation.

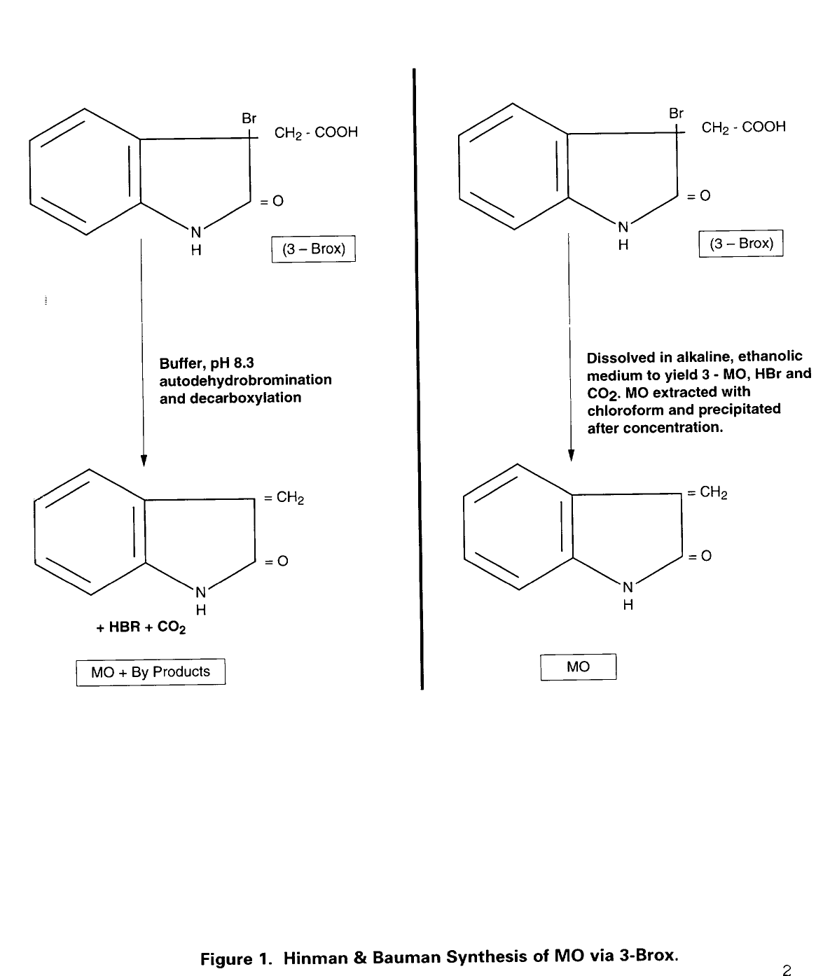

Researchers have been unable to isolate any biologically active oxidation products of IAA oxidation. Although MO, a naturally occurring oxidation product of IAA, has been synthesized, plant physiologists have always thought it to be biologically inactive. The research described by this application has found that impure MO resulting from the accepted synthetic method of Hinman and Bauman and the use of dimethylsulfoxide (DMSO) as solvent was largely responsible for the failure of plant physiologists to observe the activity of MO. The Hinman and Bauman method is identified in the Journal of Organic Chemistry, 1969. The Hinman and Bauman method calls for reaction of NBromosuccinimide with IAA to yield 3-Bromooxindole-3-acetic acid (3-Brox). In aqueous solution 3-Brox decarboxylates and dehydrobrominates to MO. Refer to FIG. 1 for flow diagram of synthesis.

FIG. 1. Hinman & Bauman Synthesis of MO via 3-Brox.

Synthesis of 3, bromooxindole-3-acetic acid and methods of obtaining 3-methyleneoxindole therefrom are given in this description. Use a 2:1 molar ratio of N-bromosuccinimide and IAA reacted in t-butanol under a stream of nitrogen. Unreacted N-bromosuccinimide is excluded with ether extraction. 3-Brox obtained from ether extract after entrapping with benzene.

Frequently, this method results in 3-Brox which is contaminated. The subsequent application of MO, derived from this synthesis, to test systems designed to ascertain auxin activity produces negative results.

The research conducted by Dr. Virendra K. Tuli has reviewed the aforementioned synthesis and found that MO is obtained by dissolving 3-Brox in an aqueous solution. In an aqueous solution, 3-Brox is dehydrobrominated and decarboxylated to 3-methyleneoxindole with the concomitant formation of bromide ions. Further research disclosed that one obtains also an unpredictable amount of tertiary-butyl-3-Brox (an ester) as well.

The presence of this compound, in turn, leads to conditions that negate the biological activity of (MO). A more reliable and predictable method of synthesis is described in this patent application under Description of Invention. Reference publication of R. L. Hinman and C. P Bauman. Reactions of N-Bromosuccinimide and Indoles. A Simple Synthesis of 3-Bromoxindoles. Jour. Org. Chem. 29: 1206 (1964) and R. L. Hinman and C. P. Bauman. Reactions of 3-Bromoxindoles. The Synthesis of 3-Methyleneoxindole. Jour. Org. Chem. 29: 2431 (1964).

### OBJECTS AND ADVANTAGES

Accordingly, several objects and advantages of my invention are the following.

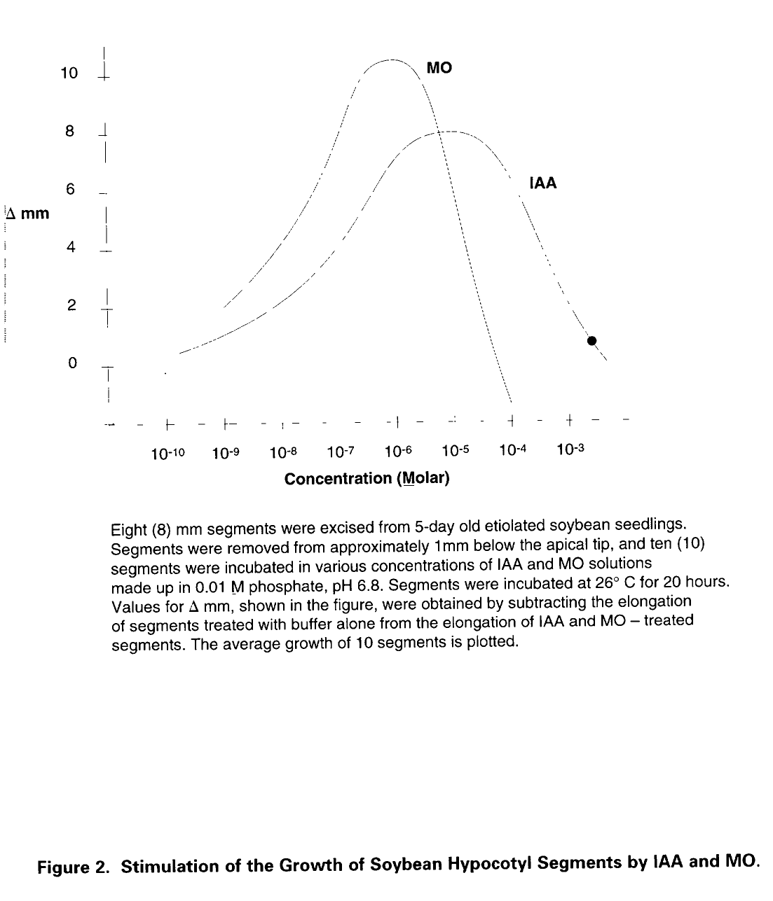

Use of MO and its solutions prepared by novel methods for stimulation of root formation in plant cuttings and as replacement for IAA or synthetic auxins in plant tissue culture. When tested against IAA, napthaleneacetic acid and indolebutyric acid, MO produced more profuse roots in a much shorter time and at significantly lower concentrations in cuttings obtained from various plants including tomatoes, begonias, African violets, roses, soybeans and peas. Refer to FIG. 2 for typical response of cuttings to test compounds and Table I for data summary.

FIG. 2. Stimulation of the Growth of Soy Bean Hypocotyl Segment by IAA and MO.

Eight (8) mm segments were excised from 3-day old etiolated wheat seedlings. Segments were removed from approximately 1 mm below the apical tip. Ten (10) segments were incubated in various molar concentrations of IAA and MO solutions made up in 0.001 M phosphate buffer. Segments were incubated at 26° C. for 20 hours. Values for Δ mm, shown in the figure, were obtained by subtracting the elongation of segments treated with buffer alone from the elongation of IAA and MO-treated segments. The average growth of 10 segments is plotted.

In terms of its efficacy as a replacement for IAA in plant tissue culture, MO was found to be far more effective than IAA as well as the synthetic auxins, napthaleneacetic acid and indolebutyric acid. Micropropagation of Nicotiana tobaccum, Ficus benjamina, Kalanchoe, and Hosta revealed that, in terms of concentration required to produce maximal growth IAA was most effective at 10⁻⁵ M in Stage II (multiplication) medium. MO produced an equivalent amount of growth at 10⁻⁸ M and maximal growth at 10⁻⁷ M.

In Stage III (rooting) medium, MO was again found to be 1,000 fold more effective than IAA and the synthetic auxins, as it was Stage II medium.

Furthermore, in plant species such as Hosta, where synthetic auxins are preferably used because of the instability of IAA, MO is far more effective than the synthetic auxins in Stage I, Stage II, and Stage III media. Photographic evidence showing the comparative response of explants to IAA and MO in Stage II and Stage III growth media is available on request. A description of the photos is provided herein.

Stage II Micropropagation.

Stage II and Stage III micropropagation of various plant species using IAA or MO as auxin source. Explants were placed in various concentrations of sterilized IAA or MO formulated in sterilized agar/mineral salts/kinnin media.

Stage III Micropropagation.

Stage III micropropagation of various plant species using IAA or MO as auxin source. Explants were placed in various concentrations of sterilized IAA or MO formulated in sterilized agar/mineral salts/kinnin media

Use of pure solutions of MO to stimulate healing in plants by aiding in the formation of callus tissue at the wound site. This would have the prophylactic effect of preventing infection in wounded trees. Application of MO in a solution, gel or lanolin paste preparation induces rapid formation of protective callus tissue. Refer to Table II for data summary. Large branches of hardwood trees were sawed off and treated with either IAA or MO prepared in lanolin paste. The final concentration of IAA and MO was 10⁻⁵M. In every test, plants treated with MO healed rapidly by forming callus tissue over the wound.

Use of pure solutions of MO in the grafting of scions to root stock. As an example, grafting of compatible, disease resistant plants to those of a good fruit bearing variety can maximize the desirable qualities of both root and scion. Application of a gel, lanolin paste or water solution promotes the fusion of different cell types to form a successful union. Continued experimentation holds the promise of using MO to facilitate the fusion of commonly accepted noncompatible fruit bearing trees, such as, grafting of lemons onto a peach tree.

TABLE I
Root Formation Rates.

| | Emergence of | *Average dry |
|---|---|---|
| | roots | wt (grams) of |
| | (No. of days | root mass (14 |
| | after | days after |
| Treatment | treatment) | treatment) |
| Control | 9 | 2 |
| IAA 10⁻⁴M | 7 | 8 |
| IAA 10⁻⁵M | 7 | 10 |
| IAA 10⁻⁶M | 9 | 7 |
| **NAA 10⁻⁴M | 8 | 5 |
| NAA 10⁻⁵M | 9 | 7 |
| NAA 10⁻⁶M | 8 | 2 |
| ***IBA 10⁻⁴M | 9 | 5 |
| IBA 10⁻⁵M | | 7 |
| IBA 10⁻⁶M | 5 | 7 |
| MO 10⁻⁶M | 4 | 11 |
| MO 10⁻⁷M | 4 | 13 |
| MO 10⁻⁸M | 6 | 10 |

Note:
*10 cuttings per treatment.
**napthaleneacetic acid
***indolebutyric acid

Legend to Table I. Ten (10) cuttings were used per treatment. Cuttings were obtained from 12-day old bean plants by excising each plant above the cotyledonery node. The cuttings were planted in vermiculite treated with various concentrations of the indicated compounds. Fourteen days after treatment, the portion of the cutting subtending the root mass was removed and dried for 48 hrs. at 50° C.

Note: Inhibition of growth at high concentrations and stimulation at low concentrations is a typical response produced by IAA. MO produces a similar response in stimulating the rooting of cuttings and the stimulation of growth of excised wheat coleoptile segments as shown in FIG. 2.

TABLE II
Effects of Various Auxins on the Formation of Callus Tissue.

| | Concentra | | | | |
|---|---|---|---|---|---|
| | tion | | | | |
| | (moles/L) | | | | |
| Auxins | 10⁻⁴ | 10⁻⁵ | 10⁻⁶ | 10⁻⁷ | 10⁻⁸ |
| IAA | 0.08 g | 0.25 g | 1.2 g | 0.80 g | 0.40 g |
| NAA | 0.06 g | 0.15 g | 0.85 g | 0.60 g | 0.20 g |
| IBA | 0.05 g | 0.18 g | 0.70 g | 0.55 g | 0.12 g |
| MO | 0.02 g | 0.15 g | 1.5 g | 1.95 g | 1.75 g |
| Control | — | 0.06 g | — | — | — |

Weight in grams of Callus tissue.

Note: Parenchyma tissue was obtained from heads of romaine lettuce. Cylinders measuring 5 mm in diameter and 20 mm in length were removed with a cork borer from the stalks of lettuce heads. The cylinders were sterilized in a 10% solution of commercial bleach (sodium hypochlorite). Three (3) mm wide circular sections were removed from the sterilized cylinders and 10 sections were placed in petri dishes containing various concentrations of auxins and MO formulated in sterilized agar/mineral salts/kinin media.

The sections were incubated at 26 degrees in the dark for seven (7) days. At the end of the incubation period, the callus tissue in each treatment was weighed and the average weight of each section was recorded. The average initial weight of the sections was 0.04 g.

### DESCRIPTION OF INVENTION

This invention relates to the oxidation products of IAA which have been found to be far more potent than IAA itself in regulating plant growth, and to a method for preparing these oxidation products. More particularly, the present invention pertains to the use of the aforementioned oxidation products, namely, HMO and MO as replacements for IAA in agricultural applications such as the rooting of cuttings and in tissue culture of plants from explants or single cells.

It is well known that at suitable concentrations, IAA can stimulate the growth of excised stem segments, promote root development in cuttings, and in combination with another group of plant growth substances known as kinins, can regenerate intact plants from single cells or meristematic tissue. The oxidation products of IAA, HMO and MO take much lesser time and concentrations to produce results similar to those produced by IAA. In accordance with the present invention, there has been discovered that the oxidation products of IAA, viz., HMO and MO, exert an action on plants that is similar to that produced by IAA. In general, HMO and MO tend to produce regulatory effects on plant growth and development at much faster rates and at concentrations that are 100 to 1000 fold lesser than IAA.

It should be noted that the auxin activity of IAA and HMO occur naturally in plants from its conversion to MO; in other words MO is an obligatory intermediate. HMO and MO occur naturally in plants from the metabolism of IAA via the oxindole decarboxylative pathway.

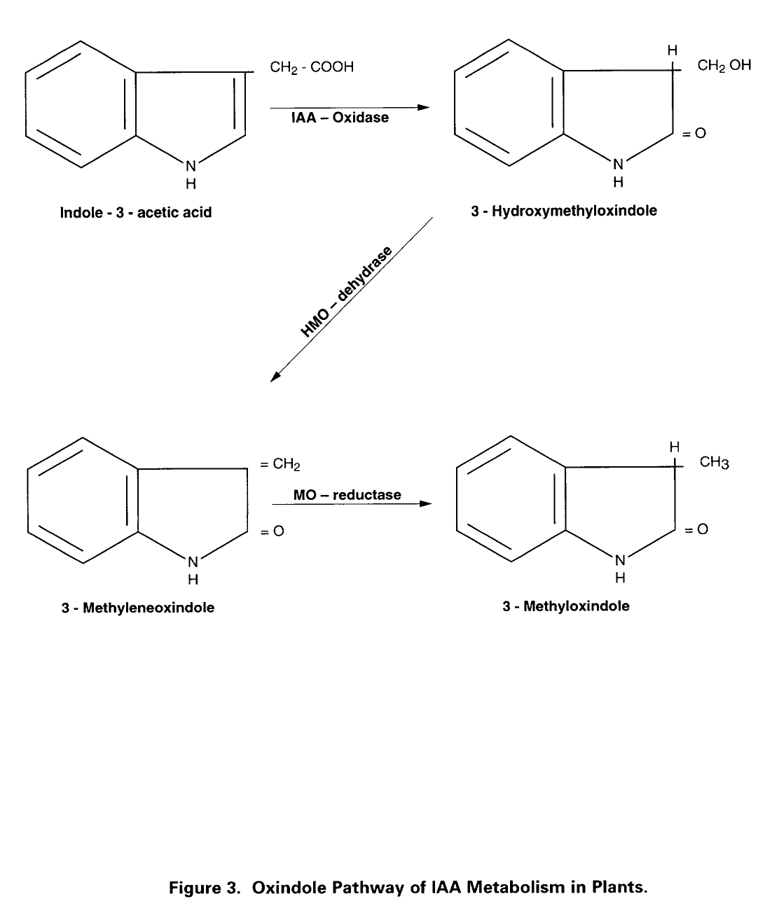

Refer to FIG. 3.

FIG. 3. Oxindole Pathway of IAA Metabolism in Plants.

The pathway describes the decarboxylative oxidation of IAA in higher plants and points out the various enzymes involved along the pathway. MO is a key intermediate of the pathway.

It has been shown that both, HMO and MO, duplicate the biological effects of IAA including the simulation of the growth of excised plant stem segments, stimulation of xylogenesis in parenchyma pith tissue obtained from lettuce or Jerusalem artichoke explants, and increased production of ethylene from plant tissue. It has also been demonstrated that MO is an obligatory intermediate in the auxin activity elicited by IAA and HMO; in other words, IAA and HMO have to be converted to MO, via the oxindole pathway, before any of the aforementioned biological effects are produced.

The present invention extends to the duplicity of the biological action of HMO and MO to include the promotion of root development In cuttings, and the ability to rejuvenate intact plants from single cells or meristemic tissue utilizing tissue culture techniques.

The invention also describes unusual, inexpensive and efficient methods to synthesize biologically active HMO and MO from the parent compound, IAA.

The new method of synthesis that is provided by the invention utilizes low concentrations of photoreactive pigments such as riboflavin, methyleneblue, rose bengal and cresol red to act as catalysts for the Photooxidation of IAA.

The pigment-catalyzed Photooxidation of IAA is carried out by weakly acidic solutions of IAA exposed to direct fluorescent or metal halide lighting.

The following examples will serve to illustrate the preferred methods for photoxidizing IAA in order to obtain the novel plant growth regulators, HMO and MO, and their exceptional ability to stimulate the growth of excised stem segments, root production in the stem cuttings, to substitute as an auxin in the micropropagation of plants, and to regenerate intact plants from meristemic tissue and isolated individual cells far more efficiently than the parent compound, IAA.

In one series of tests, the photooxidation of IAA catalyzed by various pigments in the presence of fluorescent and metal halide lights was illustrated. The objective of these tests was to establish the most ideal condition to achieve a quantified photooxidation of IAA.

Variables of exposure time to light wavelength and concentration were introduced in the sets. Table III represents the results that were observed.

In a second series of tests, the effects of the plant growth regulators of the invention upon the rooting of cuttings and regeneration of intact plants from single cells and meristemic tissue were determined. The rooting tests were carried out under standardized laboratory conditions and the regulation of intact plants from single cells and meristemic tissue was tested utilizing sterile and aseptic plant tissue culure techniques. See Table I for test data.

### OPERATION OF INVENTION

Photooxidation of IAA—Method I

The following method is a modification of previously described procedures (Phytochemical, Fukuyama, etc. ). This method was adopted because it results in a quantitative photooxidation of IAA to HMO in a relatively short time without a trace of residual IAA or other by-products. Furthermore, whereas, the previous procedures utilized riboflavin as the catalyst for the photooxidation reaction, the present investigation revealed that methylene blue, rose bengal or eosin red are as effective as riboflavin in catalyzing the photooxidation of IAA.

One-tenth mM IAA was dissolved in distilled water with slight trituration and heating. The solution was made 1 μg/ml with respect to one of the aforementioned pigments, viz., riboflavin, methylene blue, rose bengal or eosin red in a Pyrex vessel, and exposed to a bank of cool white fluorescent lights at an intensity of 1,000 ft. candles.

The photooxidation was allowed to proceed for 1.5 hours. At the end of this period, IAA was completely oxidized as there was no trace of residual IAA as determined by high performance liquid chromatography (HPLC).

The major product of the photooxidation reaction is HMO, which after incubation at room temperature for approximately 20 hours is autodehydrated to MO. This MO can be used in any of the applications described in this document.

Photooxidation of IAA—Method II

The compund 3-Brox is synthesized according to the method of Hinman and Bauman. Because of certain contaminants that are found in 3-Brox samples prepared according to the referenced procedure, it is not possible to obtain biologically active MO from the resulting 3-Brox.

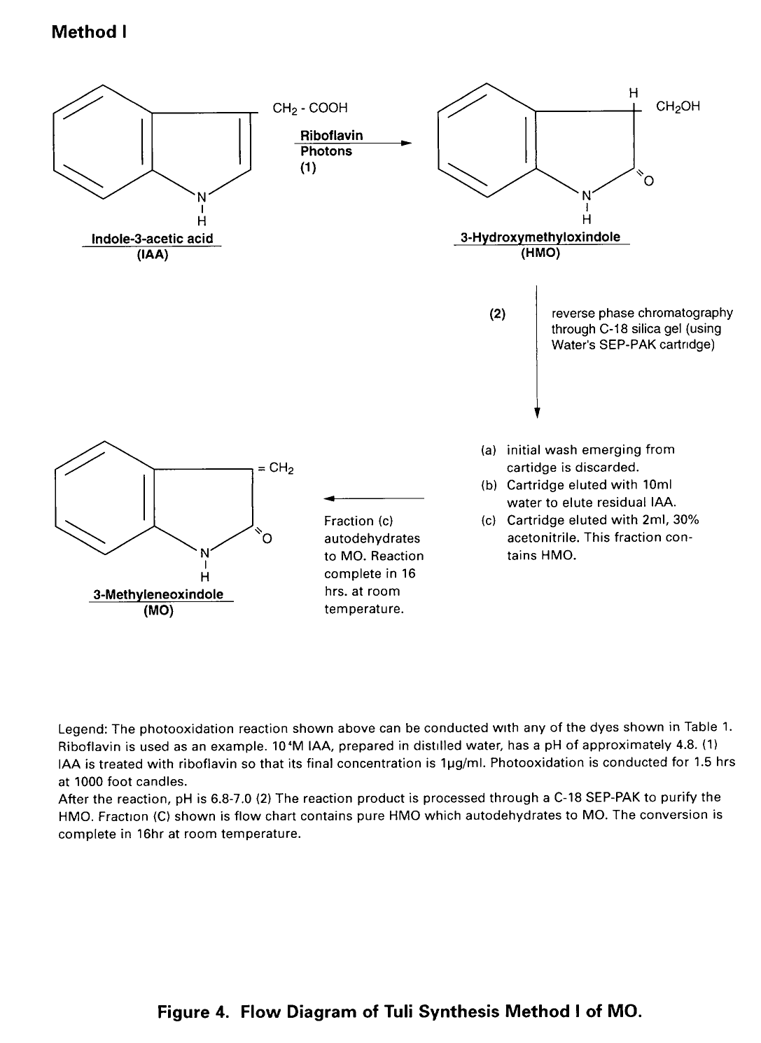

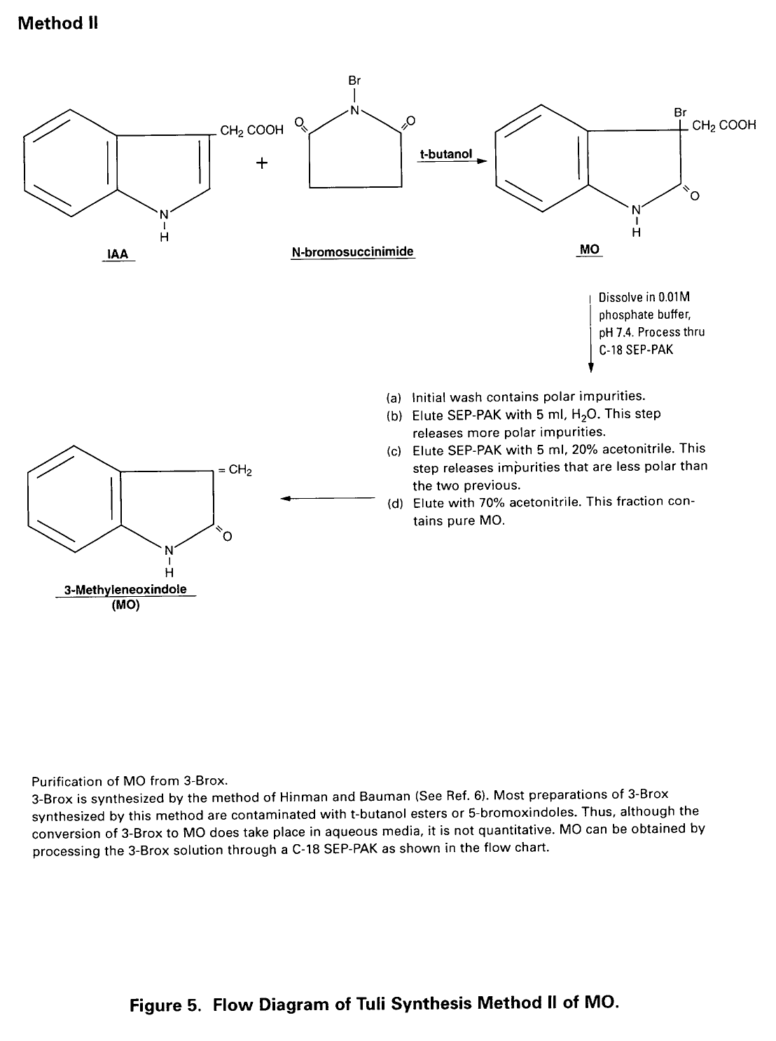

Consequently, a rapid method was developed to purify 3-Brox and obtain biologically active MO therefrom. The method used for this procedure is described herein. Refer to FIGS. 4 and 5 or flow diagrams of both synthetic methods I and II.

FIG. 4. Flow Diagram of Tuli Synthesis Method I,of MO.

Flow diagram shows Method I of obtaining pure MO from 3-Brox.

FIG. 5. Flow Diagram of Tuli Synthesis Method II, of MO.

Flow diagram shows Method II for obtaining purified MO from 3-Brox.

The compound 3-Brox was rapidly dissolved in 20 ml of 0.01 M sodium phosphate buffer, pH 7.0, to yield a 1 mM solution. The solution was passed through a C-18 SEP PAK cartridge (Water's Corp) and washed with 10 ml of water. The cartridge was subsequently eluted with 2 ml, acetonitrile: H₂O (60:40). The impurity was recovered in the first fraction, and 3-Brox was recovered in the second, less polar fraction.

The purified 3-Brox was diluted to 0.1 mM and made 0.15 mM with respect to sodium bicarbonate which caused an instant conversion to MO.

MO obtained in this manner was biologically active, and proved to be 100-1000 fold as effective as IAA in eliciting some of the physiological responses characteristically attributed to IAA.

TABLE III
Photooxidation of IAA at Varying Wavelengths

| | | | | | Time required | |
|---|---|---|---|---|---|---|
| | | | Time required | | for | |
| | | | for quantitative | | quantitative | |
| | | | oxidation | | oxidation of | |
| | | | of IAA. | | IAA. | |
| | | | Metal halide | | Fluorescent | |
| Exp. | | *μg/m | 175 W, 700 foot | | Lighting ′300 | |
| No. | Catalyst | l | candles | | foot candles | |
| 1 | Riboflavin | 10 | 35 | min. | 45 | min. |
| 2 | Riboflavin | 5 | 45 | min. | 1 | hour |
| 3 | Riboflavin | 1 | 1 | hour | 1.5 | hours |
| 4 | Methylene | 10 | 1 | hour | 1.5 | hours |
| | blue | | | | | |
| 5 | Methylene | 5 | 1.5 | hours | 1.5 | hours |
| | blue | | | | | |
| 6 | Methylene | 1 | 2.5 | hours | 1.5 | hours |
| | blue | | | | | |
| 7 | Rose bengal | 10 | 1 | hour | 1.5 | hours |
| 8 | Rose bengal | 5 | 1.5 | hours | 1.5 | hours |
| 9 | Rose bengal | 1 | 2.5 | hours | 1.5 | hours |
| 10 | Eosin red | 10 | 1 | hour | 1.5 | hours |
| 11 | Eosin red | 5 | 1.5 | hour | 1.5 | hours |
| 12 | Eosin red | 1 | 2.5 | hour | 1.5 | hours |

*μg = microgram/milliliters.
(0.5 mM IAA was used in each test)
IAA was quantitated spectrophotometrically or by the Salkowski reagent (Gardon & Weber) ref.

Conclusion, Ramifications and Scope of Invention

The research described by this specification has elucidated a metabolic pathway (FIG. 5) which leads to the formation of at least two new biologically active substances namely, HMO and MO. One or both of these substances are and have been the active regulators of growth in plants. Although IAA is widely regarded as the only naturally occurring auxin, this research shows that its oxidation products HMO and MO are also auxinogenic.

HMO and MO, intermediates of the oxindole pathway, possess auxin activity comparable to or exceeding the activity of their parent compound, IAA. Because it is unusual for several intermediates occurring in the same biochemical pathway to produce an identical physiological response, it is hypothesized herein that the auxin activity of both IAA and HMO arise from their conversion to MO. This hypothesis was based on the observation that MO is the last intermediate in the pathway to possess auxin activity.

The synthetic methods presented in this specification produce pure, biologically active forms of HMO & MO. Both methods produce equally pure products. The other synthetic methods such as, Hinman & Bauman produce contaminated products of IAA oxidation and have contributed to the hindrance of investigation and isolation of the actual auxin compounds regulating growth in all plants.

The mechanisms of action for HMO and MO are as follows:

HMO is quickly metabolized to MO which is then used by the meristematic tissues to accelerate shoot and root development. This is true for a variety of plant types and is not limited to any species.

HMO is quickly metabolized to MO which is then used by plant cuttings to rapidly initiate root development. The same occurs in explants of all species amenable to micropropagation, in stage I, stage II and stage III media.

HMO is quickly metabolized to MO which is used by the tissues at a wound site to accelerate in the formation of callus tissue to prevent infection and seal the wounded section.

HMO is quickly metabolized to MO which is used by the tissues at a site of grafting to reduce rejection of scion to root stock by stimulating new cell growth at the joint.

Although the description above contains some specific actions of the substances HMO and MO, these should not be construed as limiting the scope of the invention but as merely providing illustrations of some of the presently preferred embodiments of this invention. For example, MO may also be able to promote the union of dissimilar species in grafting and producing trees with multiple types of fruits. It is also possible that MO is a catalyst in other pathways within the cell. Perhaps it is obligatory in the production of a critical protein.

Thus the scope of the invention should be determined by the appended claims and their legal equivalents, rather than by the examples given above.

I claim:

1. A site specific method for preparation of purified HMO and MO by photooxidation of IAA. The procedure comprises the following steps.
Method
a. IAA was photooxidized by modifying the method of Fukyama (11).
b. One-tenth mM (10⁻⁴M) IAA was dissolved in distilled water with slight trituration and heating. The solution was made 1 μg/ml with respect to one of the aforementioned pigments, viz., riboflavin, methylene blue, rose bengal or eosin red in a Pyrex vessel, and exposed to a bank of cool white fluorescent lights at an intensity of 1,000 ft. candles.
c. The photooxidation was allowed to proceed for 1.5 hours. At the end of this procedure, IAA was completely oxidized as there was no trace of residual IAA as determined by high performance liquid chromatography (HPLC).
d. The major product of the photooxidation reaction is HMO, which after incubation at room temperature for approximately 20 hours is autodehydrated to MO. This MO can be used in any of the applications described in this document. Alternatively, HMO can be used immediately because it is rapidly converted to MO in plant tissues.

2. A critical modification of the method for isolation of pure MO from 3-Brox. The procedure comprises the following steps:
Method
a. 3-Brox is synthesized according to the method of Hinman and Bauman.
b. 3-Brox was rapidly dissolved in 20 ml of 0.01 M sodium phosphate buffer, pH 7.0, to yield a 1 mM solution. The solution was passed through a C-18 SEP PAK cartridge (Water's Corp) and washed with 10 ml of water. The cartridge was subsequently eluted with 2 ml, acetonitrile: H₂O (60:40).
c. The impurity was recovered in the first fraction, and 3-Brox was recovered in the second, less polar fraction.
d. The purified 3-Brox was diluted to 0.1 mM and made 0.15 mM with respect to sodium bicarbonate which caused an instant conversion to MO.
e. MO obtained in this manner was biologically active, and proved to be 100-1000 fold as effective as IAA in eliciting some of the physiological responses characteristically attributed to IAA.

3. Discovery of MO to be a biologically active compound, whose function is to operate within the plant cell as a growth hormone (auxin) or metabolic regulator.

4. Identification of HMO which is quickly metabolized to MO, which is then used by the meristematic tissues to accelerate shoot and root development. This is true for a variety of plant types and is not limited to any species. HMO is quickly metabolized to MO which is then used by plant cuttings to rapidly initiate root development. The same occurs in all plants amenable to micropropagation, in stage I, stage II and stage III media.

5. Identification of HMO which is quickly metabolized to MO, which is used by the tissues at a wound site to accelerate in the formation of callus tissue to prevent infection and seal the wounded section.

6. Identification of HMO which is quickly metabolized to MO, which is used by the tissues at a site of grafting to reduce rejection of scion to root stock by stimulating new cell growth at the joint.

7. Identification of the pathway from IAA to MO.
